(19) United States
(12) Patent Application Publication
(10) Pub. No.: US 2026/0108580 A1
(43) Pub. Date: Apr. 23, 2026

(54) METHOD FOR PREPARING BIOTIC SUPERPARAMAGNETIC NANOPARTICLES DERIVED FROM COLCHICUM RITCHII

(71) Applicant: KING SAUD UNIVERSITY, Riyadh (SA)

(72) Inventors: VEERAMANI CHINNADURAI, Riyadh (SA); KHALID A. AL-NUMAIR, Riyadh (SA); MOHAMMED A. ALSAIF, Riyadh (SA)

(21) Appl. No.: 19/028,200

(22) Filed: Jan. 17, 2025

Related U.S. Application Data
(62) Division of application No. 18/918,527, filed on Oct. 17, 2024.

(51) Int. Cl. A61K 36/88; A61K 9/51; A61K 47/02; A61P 3/04; A61P 3/10

(52) U.S. Cl. CPC A61K 36/88; A61K 9/5115; A61K 9/5192; A61K 47/02; A61P 3/04; A61P 3/10

(57) ABSTRACT

A method of preparing biotic super-paramagnetic nanoparticles can include combining a Colchicum ritchii plant extract with a ferric chloride solution to provide a mixture and adding sodium hydroxide solution to the mixture to provide the super-paramagnetic nanoparticles.

### CROSS-REFERENCE TO RELATED APPLICATION

This application is a divisional of U.S. patent application Ser. No. 18/918,527, filed on Oct. 17, 2024, the entire contents of which are incorporated herein by reference.

### TECHNICAL FIELD

The present disclosure relates to a method of preparing nanoparticles, and more particularly, to a method for preparing biotic superparamagnetic nanoparticles derived from Colchicum ritchii.

### DISCUSSION OF THE RELATED ART

Scientists are seeking new drugs derived from organic-associated nanotechnology fields for the cure or management of numerous diseases or disorders, such as obesity and diabetes, due to the enormous advantages of these drugs, including simplicity, unique effectiveness and non-toxicity.

Colchicum ritchii, also called desert saffron, is widely dispersed in Saudi Arabia and Jordan and belongs to the Colchicaceae family. Colchicum ritchii has been used to cure many diseases, such as inflammation, abdominal colics, arthritis and rheumatism. It has been reported that colchicine and homoaporphine-N-oxide alkaloids are mainly found in Colchicum ritchii. Few studies identified methods of Colchicum ritchii compound isolation and medicinal application of Colchicum ritchii.

Obesity and diabetes are major health issues faced by people in developing and developed countries alike, due to lifestyle, physical illness and genetic causes. Obesity is a complex illness and plays a vital role in causing other diseases or disorders, such as hypertension, type 2 diabetics, heart disease and certain cancer. In 2008, nearly 1.5 billion people worldwide were found to suffer from obesity. Nowadays, Saudi Arabia has one of the highest rates of obesity, affecting 7 out of 10 people. Obesity associated with type 2 diabetics is a serious health issue and is characterized by insulin resistance or inactivity. The occurrence of type 2 diabetes has increased dramatically in developing and developed countries including Saudi Arabia. Worldwide, 285 million people have been affected by diabetes in 2010 and this number is expected to increase to 439 million people by 2030, particularly in adults.

Numerous drugs are available commercially for management of obesity and diabetes having associated side effects, such as, diarrhea, skin rash, itching, and kidney complications.

Accordingly, superparamagnetic nanoparticles overcoming these shortcomings, are desired.

### SUMMARY

The present disclosure relates to a method of synthesizing biotic superparamagnetic nanoparticles using ferric chloride and Colchicum ritchii plant extract. According to an embodiment, a method of preparing biotic superparamagnetic nanoparticles can include combining Colchicum ritchii plant extract with a ferric chloride solution. In an embodiment, the biotic superparamagnetic nanoparticles are rod-shaped. In an embodiment, the biotic superparamagnetic nanoparticles have a size ranging from about 15 nm to about 24 nm.

In an embodiment, the Colchicum ritchii extract can be prepared by drying Colchicum ritchii flowers to provide dried flowers, transforming the dried flowers into a powder, e.g., by grinding, and adding the powder to boiled water. In an embodiment, ferric chloride can be dissolved in water in the presence of nitrogen to provide the ferric chloride solution.

In an embodiment, a method is provided of preparing biotic super-paramagnetic nanoparticles, comprising: combining a Colchicum ritchii plant extract with a ferric chloride solution to provide a mixture; and adding sodium hydroxide solution to the mixture to provide the super-paramagnetic nanoparticles, wherein the ferric chloride solution is obtained by dissolving ferric chloride in water in the presence of nitrogen.

According to an embodiment, the super-paramagnetic nanoparticles can be administered to a patient to treat diabetes and/or obesity.

### BRIEF DESCRIPTION OF THE DRAWINGS

The above and other features of the present disclosure will become more apparent by describing in detail exemplary embodiments thereof in conjunction with the accompanying drawings, in which:

FIG. 1 shows UV spectra demonstrating the biotic superparamagnetic nanoparticles'formation.

FIG. 2 shows transmission electron microscopy (TEM) image of the biotic superparamagnetic nanoparticles.

FIGS. 3A-3B show Dynamic Light Scattering (DLS) results of the biotic superparamagnetic nanoparticles.

FIG. 4 is a graph of Energy Dispersive X-ray (EDX) showing chemical components of biotic superparamagnetic nanoparticles.

FIG. 5 shows Fourier Transform Infrared results identifying chemical components of synthesized biotic superparamagnetic nanoparticles.

FIG. 6 is a graph showing the α-amylase inhibitory activity of biotic superparamagnetic nanoparticles compared with Metronidazole (standard drug).

FIG. 7 is a graph showing the pancreatic lipase enzyme inhibitory activity of biotic superparamagnetic nanoparticles compared with the standard drug.

### DETAILED DESCRIPTION OF THE EMBODIMENTS

Exemplary embodiments of the present disclosure will be described more fully hereinafter with reference to the accompanying drawings. The present disclosure may, however, be embodied in different forms and should not be construed as being limited to the embodiments set forth herein. Like reference numerals may refer to like elements throughout the specification. The sizes and/or proportions of the elements illustrated in the drawings may be exaggerated for clarity.

When an element is referred to as being disposed on another element, intervening elements may be disposed therebetween. In addition, elements, components, parts, etc., not described in detail with respect to a certain figure or embodiment may be assumed to be similar to or the same as corresponding elements, components, parts, etc., described in other parts of the specification.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

It will be understood by those skilled in the art with respect to any chemical group containing one or more substituents that such groups are not intended to introduce any substitution or substitution patterns that are sterically impractical and/or physically non-feasible.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of diagnosis or treatment of a condition, disorder, or disease.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

According to an embodiment, a method of preparing biotic super-paramagnetic nanoparticles can include combining a Colchicum ritchii plant extract with a ferric chloride solution to provide a mixture and adding sodium hydroxide solution to the mixture to provide the super-paramagnetic nanoparticles. The presence of the super-paramagnetic nanoparticles can be confirmed by a change in color of the mixture from brown to black. The biotic super-paramagnetic nanoparticle can be rod-shaped. A size of the biotic super-paramagnetic nanoparticle nanoparticles can range from about 15 nm to about 25 nm. In an embodiment, the super-paramagnetic nanoparticles can have a Fe core and an outer layer of Colchicum ritchii extract components.

In an embodiment, the ferric chloride solution can be obtained by dissolving ferric chloride in water in the presence of nitrogen. The ferric chloride solution can be heated to a temperature ranging from about 70° C. to about 90° C. for about 5 minutes to about 15 minutes to provide a heated solution. In an embodiment, the ferric chloride solution is heated under magnetic stirrer conditions. In an embodiment, the ferric chloride solution is heated at a temperature of about 80° C. for about 10 minutes under magnetic stirrer conditions.

In an embodiment, the Colchicum ritchii plant extract can be prepared by obtaining Colchicum ritchii plant part, e.g., fruits, seeds, leaves, roots and/or flowers, from the desert area of Saudi Arabia. In an embodiment, the Colchicum ritchii flowers can be collected, cleaned, and dried. The dried flowers can be transformed into powder using a grinder and chemical-free water. Colchicum ritchii flower (pink) tea can be prepared by combining the flower powder with water. For example, about 500 mL of boiled water can be mixed with about 5 g of the flower powder. After cooling, the flower (pink) tea can be filtered to provide the Colchicum ritchii extract.

An embodiment of the present subject matter is directed to a pharmaceutical composition comprising the biotic super-paramagnetic nanoparticles and a pharmaceutically acceptable carrier.

An embodiment of the present subject matter is directed to a method of making a pharmaceutical composition including mixing the biotic super-paramagnetic nanoparticles with a pharmaceutically acceptable carrier. For example, the method of making a pharmaceutical composition can include mixing the biotic super-paramagnetic nanoparticles under sterile conditions with a pharmaceutically acceptable carrier with preservatives, buffers, and/or propellants to create the pharmaceutical composition.

To prepare the pharmaceutical composition, the biotic super-paramagnetic nanoparticles as the active ingredient, are intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by injection, inhalation or insufflation. The biotic super-paramagnetic nanoparticles can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of the biotic super-paramagnetic nanoparticles or an amount effective to treat a disease, such as diabetes and/or diabetes, may be determined initially from the Examples described herein and adjusted for specific targeted diseases using routine methods.

The biotic super-paramagnetic nanoparticles can have antidiabetic properties. The biotic super-paramagnetic nanoparticles can be effective agents against obesity.

The biotic super-paramagnetic nanoparticles can be administered to a subject in need thereof, particularly in a therapeutically effective amount, which applies to all treatment methods described herein. In an embodiment, the biotic super-paramagnetic nanoparticles can be administered to a subject in need thereof to treat at least one disease selected from the group consisting of diabetes and obesity.

In an embodiment, the biotic super-paramagnetic nanoparticles can be administered to a subject to treat at least one disease selected from the group consisting of diabetes and obesity.

An embodiment of the present subject matter is directed to a method of treating diabetes in a subject, comprising administering to a subject in need thereof a therapeutically effective amount of the biotic super-paramagnetic nanoparticles pharmaceutical composition according to the present subject matter.

The biotic super-paramagnetic nanoparticles or a pharmaceutical compositions thereof can be administered to a subject by any suitable route. For example, the compositions can be administered orally (including bucally and sublingually), nasally, rectally, intracisternally, intra vaginally, intraperitoneally, topically, transdermally (as by powders, ointments, or drops), and/or parenterally. As used herein, “parenteral” administration refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation may also be contemplated, including, for example, embedding a composition of the disclosure in the body such as, for example, in a tissue, in the abdominal cavity, under the splenic capsule, brain, or in the cornea.

The present teachings are illustrated by the following examples.

### EXAMPLES

### Example 1

Fresh Colchicum ritchii flowers were collected from the desert area of Saudi Arabia. The whole flowers were cleaned with normal tap water followed by chemical free water. The cleaned flowers were shade dried for 3 days in an indoor environment, without using any light source. The dried flowers were transformed into powder form using a grinder and chemical-free water. Colchicum ritchii flower pink tea was prepared by adding 5 g of the flower powder to 500 mL of boiled, chemical-free water. After cooling, the flower pink tea was filtered using muslin cloth filter.

Biotic superparamagnetic nanoparticles were prepared by simple co-precipitation, i.e., by mixing Colchicum ritchii flower tea extract with a ferric chloride solutiom. Specifically, 1.10 g of ferric chloride was dissolved in 100 mL of chemical-free water underneath a nitrogen blanket. The ferric chloride solution was heated at 80° C. for 10 minutes under magnetic stirrer conditions. 20 mL of the prepared Colchicum ritchii flower tea extract was incorporated step by step, slowly, followed by addition of sodium hydroxide solution until the appearance of a black color was observed. The whole procedure was performed under warm magnetic stirring conditions. The appearance of a black color represented a visual confirmation of nanoparticle synthesis of the biotic superparamagnetic nanoparticles. The synthesized nanoparticles were cooled at room temperature and the supernatant was discarded. The pellet solutions were cleaned with chemical-free water for centrifugation at 10000 rpm for 15 mins. This cleaning process was repeated three times. Lastly, the biotic superparamagnetic nanoparticles were reduced to powder form by oven air-drying at 37° C. for 12 hrs. The black-colored powder form of the biotic super-paramagnetic nanoparticles was characterized by numerous physical and chemical methods, as follows.

### Example 2 Characterization of Biotic Superparamagnetic Nanoparticles

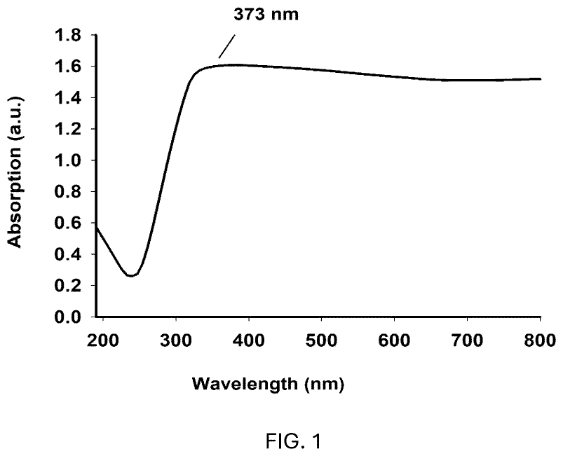

The presence of biotic super-paramagnetic nanoparticles was confirmed visually by color changes of brown to black. The biotic super-paramagnetic nanoparticles, synthesized by combining ferric chloride with Colchicum ritchii flower tea extract, were used for further characterization and confirmation by UV-spectroscopic examination. FIG. 1 shows the highest absorbance UV spectra peak at 373 nm, which demonstrates biotic superparamagnetic nanoparticles formation.

### Example 3 Tem Examination of Biotic Superparamagnetic Nanoparticles

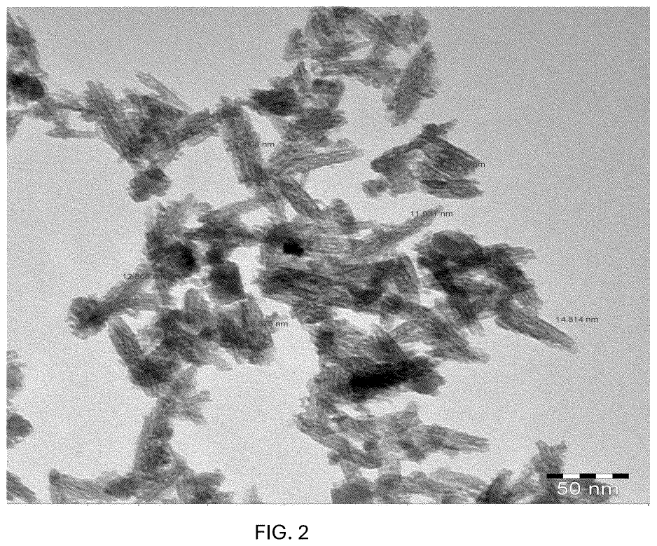

Biotic superparamagnetic nanoparticles formation was further demonstrated by TEM investigation (FIG. 2), which showed that the biotic superparamagnetic nanoparticles are rod-shaped with sizes ranging from 15.030 nm to 24.797 nm.

### Example 4 Dynamic Light Scattering (DLS) Analysis of Biotic Superparamagnatic Nanoparticles

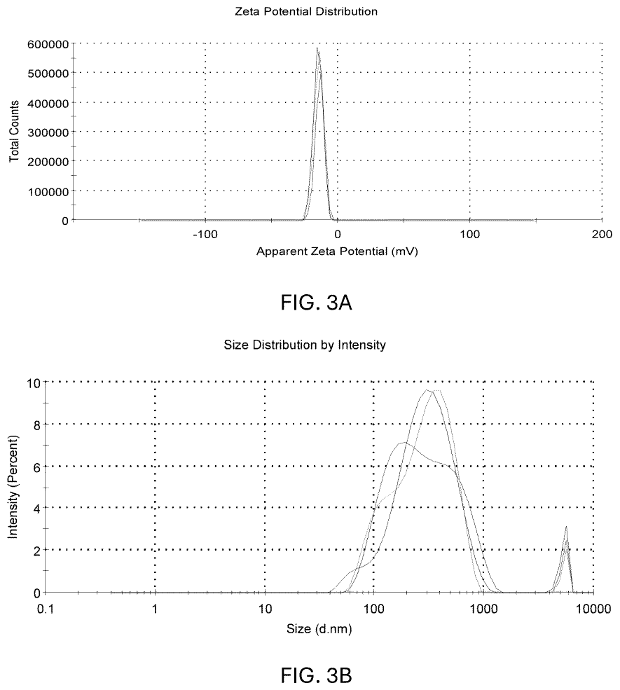

The formation of biotic superparamagnetic nanoparticles was further proven by examination of zeta potential and size distribution using Dynamic Light Scattering (DLS) methods (FIGS. 3A-3B). The biotic superparamagnetic nanoparticles peak show a very narrow size distribution and an average size value of 316.9 d. nm and zeta potential values of −13.8 mV. Hence, this observed size distribution and zeta potential peak demonstrate the formation of metallic nanoparticles.

### Example 5 Energy Dispersive X-ray (EDX) Analysis of Biotic Supermagnetic Nanoparticles

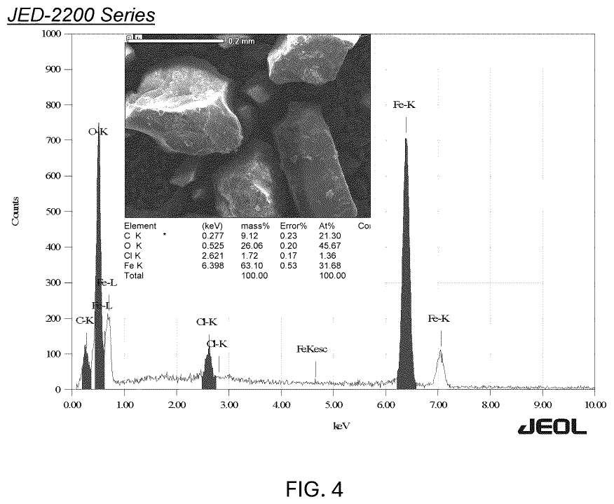

Chemical components of biotic superparamagnetic nanoparticles were identified by the Energy Dispersive X-ray (EDX) method (FIG. 4). The EDX peak of biotic superparamagnetic nanoparticles confirmed the presence of Fe as a major element as well as C, O, and Cl, which indicates iron covered with an outer layer of Colchicum ritchii flower components.

### Example 6 Fourier Transform Infrared (FTIR) Examination of Biotic Superparamagnatic Nanoparticles

Fourier Transform Infrared (FTIR) examination identified chemical components of synthesized biotic superparamagnetic nanoparticles (FIG. 5). The FTIR peak shows that the Colchicum ritchii flower biomolecules were covered with iron which demonstrates the formation of biotic superparamagnetic nanoparticles. The 3392 cm⁻¹ bands disseminated to N—H stretch of amino groups. The 1626 cm⁻¹ band is assigned to protein amide groups or to the C═O stretching vibration group. The 1384 cm⁻¹ band is assigned to the O—H stretch. The 1080 cm⁻¹ band is consigned to the C—O group. The 837, 691 and 478 cm⁻¹ bands are dispensed to the aromatic class. The presence of amine (N—H), carboxyl (—C═O) and hydroxyl (—OH) groups confirms formation of the biotic superparamagnetic nanoparticles.

### Example 7 Antidiabetic and Antiobesity Properties

α-amylase inhibitor drugs derived from natural sources are ideal for management of type-2 diabetes because of the direct action of delayed intestinal glucose absorption and lowered postprandial blood glucose.

In vitro anti-diabetic and anti-obesity properties of the synthesized biotic superparamagnetic nanoparticles were studied.

α-amylase enzyme activity: the α-amylase enzyme was assayed by an established method with slight alterations. 0.5 ml of α-amylase solution was mixed individually with different concentrations of synthesized biotic superparamagnetic nanoparticles, such as 1, 5, 10, 20, 40 and 80μg/mL and the mixtures were maintained at room temperature for 10 minutes. Secondly, 0.5 ml of starch was added kept at 37° C. for 10 minutes. Then, 1 mL of dinitrosalicylic acid was added, and the reaction was stopped. The mixture was heated at 100° C. for 5 minutes in water, then cooled at normal temperature. The absorbance was determined at 540 nm after the mixture was diluted with the 10 mL distilled water. Acarbose drug was used as a reference and the enzyme activity was calculated by the formula below:

α-amylase inhibition (%)=(A−B)/(A)×100
A signifies that the buffer added mixture instead of nanoparticles; and
B—signifies the tested sample mixture.

Pancreatic lipase is a digestive enzyme and involved in the control of overweight and obesity by digestion of dietary fat which slows adipose tissue fat deposition and suppresses weight gain. The pancreatic lipase enzyme inhibitory activity was calculated by apreviously described method with slight alterations. 10 μL of nanoparticles with different concentrations such as 1, 5, 10, 20, 40 and 80 μg/mL, of chemical-free water (negative control used to dissolve estimated nanoparticles) and positive control (Orlistat, 100 μM) were taken into corresponding wells in 96-well plate. Secondly, 40 μL of fresh porcine pancreatic lipase was mixed with the test samples, negative and positive controls and the wells were incubated at 37° C. for 15 mins. Finally, 170 μL of p-nitrophenyl butyrate (p-NPB) substrate solution was included in the wells and the wells plate was incubated at 37° C. for 25 mins. The absorbance was recorded at 405 nm.

Lipase inhibition activity was calculated as:
% Lipase inhibition=1-[X/Y]×100, where Y is a control sample absorbance and X is a test sample absorbance.

### Example 8 Anti-Diabetic and Anti-Obesity Properties

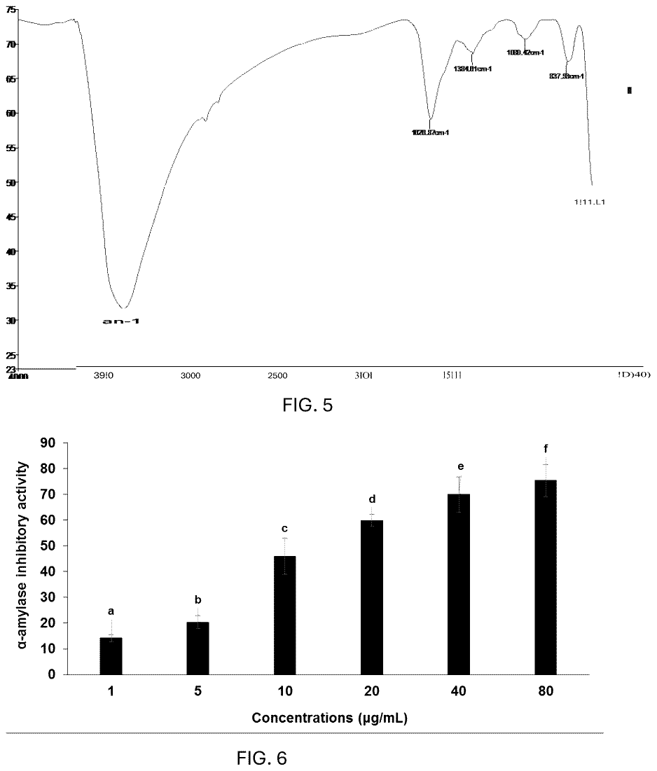

FIG. 6 shows the α-amylase inhibitory activity of biotic superparamagnetic nanoparticles and the results were compared with Metronidazole (standard drug). The biotic superparamagnetic nanoparticles showed significantly increased α-amylase inhibitory activity in a dose dependent manner. This inhibitory activity was comparable to that of the standard drug. Hence, our study demonstrated that the biotic superparamagnetic nanoparticles have anti-diabetic potential activity through the inhibitory effect of α-amylase.

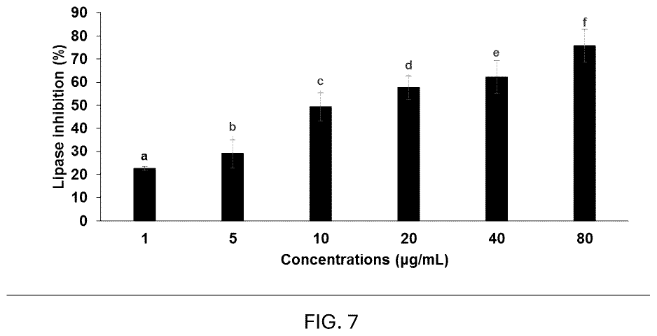

FIG. 7 shows the pancreatic lipase enzyme inhibitory activity of biotic superparamagnetic nanoparticles and the results were compared with the standard drug. The biotic superparamagnetic nanoparticles showed significantly increased pancreatic lipase enzyme inhibitory activity with the dose dependent manner. This inhibitory activity was comparable to the standard drug. Hence, our study demonstrated that the biotic superparamagnetic nanoparticles demonstrated anti-obesity activity through the inhibitory effect of pancreatic lipase enzyme.

While the present disclosure has been particularly shown and described with reference to exemplary embodiments thereof, it will be apparent to those of ordinary skill in the art that various changes in form and detail may be made therein without departing from the spirit and scope of the present disclosure as defined by the following claims.

What is claimed is:

1. A method of treating obesity in a subject, comprising:
administering a therapeutically effective amount of biotic super-paramagnetic nanoparticles to a subject in need thereof; wherein the biotic super-paramagnetic nanoparticles are prepared according to a method comprising:
combining a Colchicum ritchii plant extract with a ferric chloride solution to provide a mixture; and
adding sodium hydroxide solution to the mixture to provide the super-paramagnetic nanoparticles.

2. The method of claim 1, wherein the Colchicum ritchii plant extract is derived from the Colchicum ritchii flower.

3. The method of claim 1, wherein the ferric chloride solution is obtained by dissolving ferric chloride in water in the presence of nitrogen.

4. The method of claim 1, wherein the ferric chloride solution is heated to a temperature ranging from about 70° C. to about 90° C. for about 5 minutes to about 15 minutes to provide a heated solution.

5. The method of claim 4, wherein the ferric chloride solution is heated under magnetic stirrer conditions.

6. The method of claim 4, wherein the ferric chloride solution is heated at a temperature of about 80° C. for about 10 minutes under magnetic stirrer conditions.

7. A method of treating obesity in a subject, comprising:
administering a therapeutically effective amount of biotic super-paramagnetic nanoparticles to a subject in need thereof; wherein the super-paramagnetic nanoparticles are prepared according to a method comprising:
combining a Colchicum ritchii plant extract with a ferric chloride solution to provide a mixture; and
adding sodium hydroxide solution to the mixture to provide the super-paramagnetic nanoparticles, wherein the ferric chloride solution is obtained by dissolving ferric chloride in water in the presence of nitrogen.

8. The method of claim 7, wherein the ferric chloride solution is heated to a temperature ranging from about 70° C. to about 90° C. for about 5 minutes to about 15 minutes to provide a heated solution.

9. The method of claim 8, wherein the ferric chloride solution is heated under magnetic stirrer conditions.

10. The method of claim 8, wherein the ferric chloride solution is heated at a temperature of about 80° C. for about 10 minutes under magnetic stirrer conditions.

11. The method of claim 7, wherein the Colchicum ritchii plant extract is derived from the Colchicum ritchii flower.
